(19) United States
(12) Patent Application Publication
(10) Pub. No.: US 2013/0142894 A1
(43) Pub. Date: Jun. 6, 2013

(54) HEALTH SUPPLEMENT COMPOSITIONS FOR COMPANION ANIMALS AND METHODS OF USING THE SAME

(71) Applicant: Bimini LLC, Rancho Santa Fe, CA (US)

(72) Inventor: Samer Al-Murrani, Topeka, KS (US)

(73) Assignee: BIMINI LLC, Rancho Santa Fe, CA (US)

(21) Appl. No.: 13/679,629

(22) Filed: Nov. 16, 2012

Related U.S. Application Data
(60) Provisional application No. 61/560,476, filed on Nov. 16, 2011.
(60) Provisional application No. 61/589,743, filed on Jan. 23, 2012.
(60) Provisional application No. 61/651,473, filed on May 24, 2012.

(51) Int. Cl. A61K 36/76; A61K 36/481; A61K 36/64

(52) U.S. Cl. CPC A61K 36/76; A61K 36/64; A61K 36/481
USPC 424/769; 424/725

(57) ABSTRACT

Aspects of the present invention include compositions configured for maintaining health and/or treating progressive degenerative conditions, e.g., hip and joint conditions, kidney conditions, etc., in companion animals. The compositions include supplement formulations and a highly palatable edible backbone, e.g., in the form of a soft chew, tablet or drink. Supplement formulations of interest include one or more naturally occurring active agents which are effective in maintaining health and/or treating a progressive degenerative condition. Methods of maintaining health of and/or treating a companion animal for a progressive degenerative disease by administering the supplement formulations are also provided.

### CROSS-REFERENCE TO RELATED APPLICATIONS

Pursuant to 35 U.S.C. §119 (e), this application claims priority to the filing date of U.S. Provisional Patent Application Ser. No. 61/560,476 filed Nov. 16, 2011; U.S. Provisional Patent Application Ser. No. 61/589,743filed Jan. 23, 2012 and U.S. Provisional Application No. 61/651,473 filed May 24, 2012; the disclosures of which applications are herein incorporated by reference.

### INTRODUCTION

There are many progressive degenerative diseases (PDGs) that affect almost every organ in the body. Some are pathological in nature, others are a natural consequence of the aging process. Pets like humans also suffer from PDGs and two of the most prevalent are Chronic Kidney (Renal) Disease (CKD) and Degenerative Joint Diseases (DJD) affecting the hips and joints. While CKD is more prevalent and is diagnosed more readily in the cat population, DJD of the hip and joint is far more readily diagnosed in the dog population.

Chronic Renal or Kidney Disease (CKD) in cats has an incidence of 1.6% of the total cat population (˜1.4 million cats in the US alone) and 0.9% of the total dog (˜675,000 dogs in the US alone) (see Veterinary Medical Data Base, University of Purdue). Indeed, renal failure is one of the most common causes of death in dogs. In animals which suffer from renal disease, several blood indices are used to determine the severity, including blood urea nitrogen (BUN) and creatinine, which levels increase in the bloodstream during the course of renal failure because damage to the kidney of the animal reduces its ability to adequately filter waste products. Because inadequate filtration of waste products is the fundamental basis of renal disease, BUN and creatinine are considered the primary indicators of renal disease.

Numerous risk factors are associated with renal disease. Age and breed are clear risk factors for dog and cat species. According to the International Renal Interest Society (IRIS), almost every cat over 9 years of age will exhibit some form of kidney disease. Also according to IRIS, renal disease initiation factors can include: Glomerular diseases which are a leading cause of renal disease in dogs; diseases such as infectious diseases, neoplasia, inflammatory diseases (which can be primary, or secondarily associated with renal disease); tubulointerstitial diseases, which are a common cause of CKD in cats (a specific relationship between this type of CKD and urinary tract infections, stones, obstructions and toxic drugs has been reported in humans); clinical factors including sequelae of acute renal failure, urinary tract infections, urinary stones, obstruction of lower urinary tract, diabetes, hypercalcemia, and heart failure; drugs such as aminoglycosides, sulfonamides, polymyxin B, amphotericin and chemotherapeutic agents; and other progression factors such as high blood pressure, high level of proteinuria, and poor glycemic control in diabetes mellitus.

Dogs unlike most other species have undergone a large degree of selective breeding, this adds to the regular wear and tear associated with the aging process itself rendering some breeds of dogs more prone to changes that come with age faster. Mobility is a perfect example of this where older dogs that are also from larger breeds may exhibit hip and joint and overall mobility problems earlier than others. According to the Arthritis Foundation some of the classic signs of hip and joint problems in dogs include:

Favoring a limb

Difficulty sitting or standing

Sleeping more

Seeming to have stiff or sore joints

Hesitancy to jump, run or climb stairs

Weight gain

Decreased activity or less interest in play

Attitude or behavior changes

Being less alert

Therapies for dogs may include:
Healthy diet and exercise to help maintain proper weight.
Working with a veterinarian to find a drug tabletment that helps relieve the pain.
Nonsteroidal anti-inflammatory drugs (NSAIDS): the most common form of pharmaceutical tabletment for arthritis in dogs.
Over-the-counter pet tabletments, such as pills or food containing either glucosamine and chondroitin sulfate or Omega fatty acids. Both have shown to help relieve the symptoms of arthritis in dogs.
A veterinarian-prescribed NSAID and an over-the-counter tabletment that together may help decrease pain and disease progression.
Medications designed for humans should never be given to dogs or cats. Certain medications can be toxic to pets—particularly acetaminophen, ibuprofen and aspirin—and a safe dose will differ between a given breed, e.g., greyhound and a dachshund or species.

### SUMMARY

Aspects of the present invention include compositions configured for maintaining health and/or treating progressive degenerative conditions, e.g., hip and joint conditions, kidney conditions, etc., in companion animals. The compositions include supplement formulations and a highly palatable edible backbone, e.g., in the form of a soft chew, tablet or drink. Supplement formulations of interest include one or more naturally occurring active agents which are effective in maintaining health and/or treating a progressive degenerative condition. In certain aspects, the supplement formulations include an amount of one or more actives effective in promoting healthy hips and joints, e.g., agents that exhibit anti-inflammatory and/or pain relief activity, such as willow and/or cat's claw, or in promoting healthy kidney function, e.g., agents that exhibit anti-inflammatory and/or hypotensive activity Rehmannia and/or Astragalus. Methods of treating a companion animal for a progressive degenerative disease, e.g., methods of supporting or promoting the renal health and hip and joint health, of a companion animal by administering the supplement formulations to the companion animal are also provided.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

As used herein, the term “companion animal” refers to a domesticated mammalian species that serves as a companion, or pet, to a human. This includes numerous domesticated mammals, including dogs, cats, horses, mice, rats, guinea pigs, rabbits, ferrets, and the like. Companion animals may vary in weight, ranging in some instances from 0.25 lbs. to 2500 lbs., such 2 lbs. to 2000 lbs. For example, where the companion animal is a dog, the weight of the dog may vary, and in some instances ranges from 0.25 to 500 lbs., such as 1 to 4001bs., including 5 to 350 lbs. including 30 to 300 lbs. Where the companion animal is a cat, the weight of cat may vary, and in some instances ranges from 1 to 50 lbs., such as 5 to 30 lbs., including 7.5 to 25 lbs. Where the companion animal is a horse, the weight of the horse may vary, and in some instances ranges from 50 to 2500 lbs., such as 500 to 2250 lbs., including 750 to 2200 lbs.

As used herein, the term “highly palatable backbone” refers to an ingestible composition for a companion animal that is in a form that the animal enjoys or is desirous of ingesting and rarely, if ever, is rejected due to its unpleasant taste, shape, texture, hardness/softness, odor, color, size or other property.

As used herein, the term “active” or “actives” refers to a component of a supplement formulation that provides a specific activity or has a desired effect when ingested. Active agents of interest include naturally occurring active agents, e.g., active agents found in nature. An active may be one or more whole, raw, processed, or minimally-processed component, such as a herb or a particular part of a herb, e.g., the dried root of a herb, or an active may be a more processed form of one or more component, such as an extract, a decoction, an isolated or purified fraction, etc. No limitation in this regard is intended. In some cases, an active (or actives) includes or is derived from multiple different starting components, e.g., an active may be a mixture of two, three, four or more, etc., different herbs. In addition, the active(s) employed to manufacture a single supplement formulation can be in different forms, e.g., a whole, raw, processed, or minimally-processed form.

As used herein, the term “Willow bark” refers to the following species of herbs: Salix fragilis (Crack Willow), Salix purpurea (Purple Willow), Salix daphnoides and Salix Alba (While Willow). Common names include: White Willow bark, Willow, Salix Cortex, and any combination thereof. The part of the plant most commonly used is the inner bark (also outer) and bark from the branches. Salix contains phenolic glycosides, including salicin, salicortin, salireposide, picein and triandrin. Up to 20% of Salix bark consists of Tannins. Salix also contains catechins and flavonoids. The indications for willow bark include but are not limited to anti-inflammatory, anti-pyretic and analgesic.

1) Akao T, Yoshino T, Kobashi K. and Hattori M. Evaluation of salicin as an antipyretic prodrug that does not cause gastric injury. Planta Med 2002, 68:714-718

2) Chrubasik S., Eisenberg, E., Balan E., Weinberger T., Luzzati R. and Conradt C. Tabletment of low back pain exacerbations with willow bark extract: A randomized double blind study. Am. J. Med. 2000, 109:9-14.

3) Chrubasik S., Kunzel, O., Model A., Conradt C and Black A. Tabletment of low back pain with a herbal or synthetic anti-rheumatic: A randomized controlled study: Willow bark extract for low back pain. Rheumatology 2001, 40:1388-1393.

As used herein, the term “Cat's claw” refers to the Uncaria Tomentosa species of herbs. Similar species include Uncaria rhynchophylla and Uncaria guiaensis. Common names include: Una de gato, samento, garabato Colorado. The plant exists in two chemotypes, one that is high in pentacyclic alkaloids and the other is high in tetracyclic alkaloids. In certain embodiments one or the other chemotype is used. Cat's claw refers to any part of the herb, including the inner bark of the stem or root. In certain embodiments, only one part of the herb is employed, e.g., the root. Constituents include: Indole alkaloids, triterpene, saponins and flavonoids. Some of the herb's actions include but are not limited to anti-inflammatory, anti-rheumatic and immune modulatory actions.

1) Mur, E., Hartig, F., Eibl, G. and Shirmer M. Randomized double blind trial of an extract from the pentacyclic alkaloid-chemotype of Uncaria Tomentosa for the tabletment of rheumatoid arthritis. J. Rheumatol 2002, 29:678-681.

2) Piscoya J., Rodriguez Z., Bustamante S. A., Okuhama N. N., Miller M. J. and Sandoval M. Efficacy and safety of freeze-dried cat's claw in osteoarthritis of the knee: mechanisms of action of the species Uncaria guianensis. Inflamm. Res. 2001, 50:442-448.

As used herein, the term “Rehmannia” refers to Rehmannia species of herbs, including Rehmannia glutinosa, Rehmannia chingii, Rehmannia elata, Rehmannia henryi, Rehmannia piasezkii, Rehmannia solanifolia, and any combination thereof. Rehmannia refers to any part of the herb, including the flowers, leaves, stems, roots, etc. In certain embodiments, only one part of the herb is employed, e.g., the root. Rehmannia has anti-inflammatory and diuretic actions and contains iridoids (such as ajugol and rehmanniosides), phenylethanoid glycosides (such as verbascoside and echinacoside), sugars and sterols. References describing Rehmannia include the following:

1) Kang, D. G., Sohn, E. J., Moon, M. K., Lee Y. M., Lee, H. S. Rhemannia glutinosa ameliorates renal function in the ischemia/reperfusion-induced acute renal failure rats. Biol Pharm Bull 2005, 28:1662-1667;

2) Yokozawa, T., Kim, H.Y., Yamabe, N. Amelioration of diabetic nephropathy by dried rhemannia Radix (Di Huang) extract. Am J. Chin Med 2004, 32:829-839; and

3) Su Z. Z., He, Y. Y., Chen G. [Clinical and experimental study on effects of man-shen-ling oral liquid in the treatment of 100 cases of chronic nephritis] Zhongguo Zhong Xi Yi Jie He Za Zhi 1993, 13:269-272, 259-260.

4) Albach, Li, Zhao, and Jensen “Molecular systematics and phytochemistry of Rehmannia (Scrophulariaceae)” Biochemical Systematics and Ecology, May 2007, Volume 35, Issue 5, Pages 293-300.

As used herein, the term “Astragalus” refers to the Astragalus species of herbs, including Astragalus membranaceus, Astragalus floridus, Astragalus tonglensis, Astragalus propinguus, Astragalus chrysopterus, and any combination thereof. Astragalus refers to any part of the herb, including the flowers, leaves, stems, roots, etc. In certain embodiments, only one part of the herb is employed, e.g., the root. Astragalus has many hypotensive as well as renal protective functions and contains triterpene saponins (astragalosides I-X and isoastragalosides I-IV) and polysaccharides (such as astragalan and astraglucan). References describing this herb include the following:

1) Change, H and But, P. Pharmacology and applications of chinese material medica, vol. 2 Singapore: World Scientific, 1987;

2) Yin, X., Zhang, Y., Wu, H., et al. Protective Effects of Astragalus Saponin I on early stages of diabetic nephropathy in rats. J. Pharmacol. Sci 2004, 95:256-266;

3) Peng, A., Gu, Y., Lin, S. Y. Herbal treatment for renal disease. Ann Acad Med Singapore 2005, 34:44-51; and

4) Modern TCM pharmacology, 1997.

### Methods and Compositions

As summarized above, aspects of the present invention include compositions and methods for maintaining health and/or treating a companion animal for a progressive degenerative condition. Accordingly, methods and compositions are provided for supporting health of a companion animal, i.e., e.g., for keeping the animal in a health state, for improving the health of an otherwise healthy animal, etc. Methods and compositions are also provided for treating a companion animal for a progressive degenerative condition. As reviewed above, progressive generative conditions include conditions or diseases which are pathological in nature, conditions that are a natural consequence of the aging process, etc. Specific examples of progressive degenerative conditions include, but are not limited to: Chronic Kidney (Renal) Disease (CKD), Degenerative Joint Diseases (DJD) affecting the hips and joints, etc.

Aspects of the invention include administering to a companion animal a composition that is configured or formulated for maintaining health and/or treating a progressive degenerative condition, e.g., hip and joint condition, kidney conditions, etc. Compositions of interest include supplement formulations and a highly palatable edible backbone, e.g., in the form of a soft chew, tablet or drink. Supplement formulations of interest include one or active agents which are effective in maintaining health and/or treating a progressive degenerative condition. As reviewed above and in greater detail below, active agents of interest include naturally occurring active agents. In certain aspects, the supplement formulations include an amount of one or more actives effective in promoting healthy hips and joints, e.g., agents that exhibit anti-inflammatory and/or pain relief activity, such as willow and/or cat's claw, or in promoting healthy kidney function, e.g., agents that exhibit anti-inflammatory and/or hypotensive activity, such as but not limited to Rehmannia and/or Astragalus.

Accordingly, aspects of the invention include compositions that include a supplement formulation and a highly palatable edible backbone, e.g., in the form of a soft chew, tablet or drink. Methods of promoting the hip and joint health or renal function of a companion animal by administering the compositions to the companion animal are also provided. The compositions may be administered to healthy companion animals as a preventative measure against deterioration of hip and joint or renal functions.

Aspects of the present invention include methods for treating, preventing, ameliorating or modifying a DJD or dysfunction of the corresponding tissues or CKD or dysfunction of the corresponding tissues in a companion animal that includes administering the companion animal a supplement composition as described herein for a sufficient period of time to treat, prevent, ameliorate or modify DJD, CKD or dysfunction of the corresponding tissues in the companion animal. In certain embodiments, the supplement formulation present in the compositions includes an effective amount of one or more actives that promote healthy hip and joint and healthy renal function, where in certain embodiments, the active is selected from Willow bark and/or Cat's claw for hip and joint health and from Rehmannia and/or Astragalus for renal health. In certain embodiments, only one part of the herb is used for the active component, e.g., the root. The term “effective amount” means an amount that, when ingested by a companion animal (e.g., according to a dosage regimen over a sufficient period of time), is sufficient to improve one or more clinical signs or symptoms of DJD or CKD or dysfunction of the corresponding tissues and/or reduce or eliminate one or more risk factor associated with DJD or CKD or dysfunction of the corresponding tissues, thereby promoting hip and joint or kidney health. Thus, aspects of the present invention utilize a highly palatable formulation composition which, when administered to a companion animal, such as a dog or cat or a horse, results in an improvement in one or more clinical signs, symptoms, or risk factors associated with DJD or CKD or dysfunction of the corresponding tissues.

In practicing these methods, a companion animal may be examined, for example by a veterinarian or other qualified individual, for one or more specific physical or physiological parameters that are indicative of an active DJD (e.g., Arthritis) or CKD or that the animal is at risk of developing a DJD or CKD (also called a risk factor) prior to administering the supplement formulation composition to the companion animal. See the introduction for a partial list of DJD and CKD indicators/risk factors.

As but one example, a companion animal may be assessed for specific plasma, urinary and/or endocrine variables to determine whether the animal should be started on a supplement formulation regimen according to aspects of the present invention. The animal may also be subjected to radiological examination to determine if any of the joints appear to be calcified. Furthermore, if the companion animal, for example, is found to have a variable outside the normal range it may be started on a supplement formulation regimen according to aspects of the present invention. Thus, aspects of the present invention include testing an animal for one or more radiological tests and/or physical or physiological parameter/variable and starting the companion animal on a supplement formulation regimen as described herein when one or more parameter/variable is outside the normal range. Normal ranges for such variables can be readily determined by one of skill in the art. Exemplary ranges for certain variables are reproduced below (from Plumb's Veterinary Drug Handbook 6^{th} Edition, Plumb 2008, incorporated herein by reference). (Note that the normal range for Glucose in Canine should read 75-145 mg/dl, not 75-45 mg/dl as shown below). Additional exemplary normal ranges for variables can also be found in Plumb's Veterinary Drug Handbook 7th Edition (Plumb, 2011; incorporated herein by reference); Marshfield Labs (see, e.g., http://www.marshfieldlabs.org/marshfieldlabs/default.aspx); and VCA Antech (see, e.g., http://www.vcaantech.com).

Reference Laboratory Ranges
Note: The following reference ranges are as a general reference only;
refer to the “normals” for the laboratory you are using

| | | | | | |
|---|---|---|---|---|---|
| Test | Units | Canine | Feline | Bovine | Equine |

Chemistry: Canine, Feline, Bovine, Equine
Values are from: Marshfield Clinic Laboratories, Veterinary Diagnostic Service; 2007

| | | | | | |
|---|---|---|---|---|---|
| Glucose | mg/dl | 75-45 | 56-153 | 50-79 | 52-121 |
| AST (SGOT) | U/L | 13-81 | 14-54 | 57-108 | 156-597 |
| ALT (SGPT) | U/L | 14-151 | 26-128 | 11-47 | 3-60 |
| Alkaline phosphatase | U/L | 13-289 | 14-102 | 26-78 | 86-262 |
| Total bilirubin | mg/dL | 0.1-0.5 | 0.0-0.2 | 0.1-0.4 | 0.4-3.3 |
| Cholesterol | mg/dL | 98-300 | 71-218 | 112-331 | 59-125 |
| Total protein | gm/dL | 5.0-8.3 | 5.9-8.4 | 6.3-8.5 | 5.2-8.2 |
| Albumin | gm/dL | 2.6-4.0 | 2.3-3.9 | 3.2-4.3 | 2.8-3.8 |
| Urea nitrogen | mg/dL | 8-30 | 18-36 | 8-22 | 9-27 |
| Creatinine | mg/dL | 0.5-2.0 | 0.6-2.0 | 0.6-1.4 | 0.4-1.9 |
| Phosphorus | mg/dL | 2.5-7.9 | 2.7-7.5 | 4.4-9.2 | 1.7-5.8 |
| Calcium | mg/dL | 8.7-12.0 | 8.7-11.7 | 7.9-10.5 | 10.2-13.4 |
| Sodium | mmol/L | 141-159 | 146-160 | 140-151 | 130-144 |
| Potassium | mmol/L | 3.4-5.6 | 3.3-5.4 | 3.7-5.6 | 2.9-5.6 |
| Chloride | mmol/L | 100-118 | 110 [symbol] 123 | 100-109 | 92-107 |
| Bicarbonate | mmol/L | 16-31 | 15-24 | 22-29 | 21-33 |
| CK | U/L | 50-554 | 55-688 | 50-271 | 96-620 |
| GGT | U/L | 3-19 | 0-5 | 12-30 | 5-51 |
| Magnesium | mg/dL | 1.5-3.4 | 2.0-2.8 | 1.8-2.9 | 1.6-2.3 |
| Amylase | U/L | 268-1653 | 422-1328 | * | 1-10 |
| Lipase | U/L | 81-696 | 8-289 | * | 14-50 |
| SDH | U/L | 0.7-20.0 | 0.0-10.9 | 12.2-46.0 | 2.7-8.3 |
| LDH | U/L | 19-396 | 52-331 | 806-1250 | 151-776 |
| Triglycerides | mg/dL | 18-248 | 17-133 | * | 10-61 |
| T4 | μg/dL | 0.8-4.0 | 1.9-4.8 | 2.8-7.0 | 2.5-4.8 |
| Bile acid, fasting | μmol/L | 0.0-12.0 | 0.0-5.0 | 0.0-12.0 | 4.6-13.3 |
| Bile acid, postprandial | μmol/L | 0.0-25.0 | 5.0-15.0 | 0.0-12.0 | * |
| Fructosamine | μmol/L | 181-400 | 172-370 | * | 232-365 |
| Iron | μg/dL | 46-214 | 50-141 | * | 89-262 |
| BHBA | mg/dL | 0.7-3.2 | 0.1-4.6 | 0.4-8.8 | * |
| Uric acid | mg/dL | 0.1-1.4 | 0.0-0.5 | 0.6-1.7 | 0.1-0.6 |
| Direct bilirubin | mg/dL | 0.0-0.2 | 0.0-0.2 | 0.0-0.2 | 0.0-0.2 |

Hematology: Canine, Feline, Bovine, Equine
Values are from: Marshfield Clinic Laboratories, Veterinary Diagnostic Service; 2007

| | | | | | |
|---|---|---|---|---|---|
| Red Blood Count (RBC) | ×10⁶/μL | 4.48-8.53 | 5.8-11.0 | 5.0-10.0 | 5.63-12.09 |
| Hemoglobin | g/dL | 10.5-20.1 | 8.0-16.0 | 8.0-15.0 | 9.8 [symbol] 17.1 |
| Hematocrit | % | 33.6-58.7 | 28.0-47.0 | 24.0-46.0 | 27.0-47.5 |
| Mean corp. vol. (MCV) | fL | 63.0-78.3 | 37.7-50.0 | 40.0-60.0 | 33.5-55.8 |
| Mean corp. Hgb (MCH) | pg | 15.3-39.2 | 12.3-17.2 | 11.0-17.0 | 12.2-19.3 |
| Mean corp. Hgb conc. | g/dL | 30.8-35.9 | 31.1-36.0 | 30.0-36.0 | 32.4-37.4 |
| (MCHC) | | | | | |
| Red cell dis. width (RDW) | % | 13.4-18.1 | 17.0-24.0 | 26.0-30.0 | 20.6-29.0 |
| Platelet count | ×10³/μL | 110-460 | 160-660 | 230-690 | 95-285 |
| White blood count (WBC) | ×10³/μL | 4.0-17.6 | 3.7-20.5 | 4.0-12.0 | 4.1-14.3 |
| Segmented neutrophil | ×10³/μL | 2.5-14.3 | 1.3-15.7 | 0.6-4.0 | 1.7-10.4 |
| absolute no. | | | | | |
| Banded neutrophil | ×10³/μL | 0.0-0.2 | 0.0-0.5 | 0.0-0.12 | 0.0-0.1 |
| absolute no. | | | | | |
| Lymphocyte absolute no. | ×10³/μL | 0.3-3.9 | 1.0-7.9 | 2.5-7.5 | 0.6-6.7 |
| Eosinophil absolute no. | ×10³/μL | 0.0-1.3 | 0.1-2.0 | 0.0-2.4 | 0.0-0.5 |
| Basophil absolute no. | ×10³/μL | 0.0-0.1 | 0.0-0.1 | 0.0-0.2 | 0.0-0.2 |

Coagulation: Canine, Feline, Bovine, Equine
Values are from: Marshfield Clinic Laboratories, Veterinary Diagnostic Service; 2007

| | | | | | |
|---|---|---|---|---|---|
| Antithrombin III | % | 84.0-128.0 | 87.0-143.0 | * | * |
| APTT (Activated Partial | seconds | 9.1-15.6 | 9.9-23.4 | 21.3-35.8 | 33.0-55.0 |
| Thromboplastin Time | | | | | |
| D-Dimer | μg/mL | 0.0-0.4 | 0.0-0.4 | * | * |
| Fibrin split products | μg/mL | 0.0-4.0 | 0.0-4.0 | * | * |
| Fibrinogen, Semi- | mg/dL | * | * | 300-700 | 100-400 |
| quantitative | | | | | |
| PIVKA | seconds | 12.0-18.0 | 19.0-33.0 | * | 17.0-23.0 |
| PT (Prothrombin time) | seconds | 5.4-8.8 | 7.2-12.5 | 16.8-20.7 | 9.1-12.6 |

Urinalysis: Canine, Feline

| | | | |
|---|---|---|---|
| Specific Gravity | | 1.001-1.070 | 1.001-1.080 |
| pH | | 5.5-7.5 | 5.5-7.5 |
| Volume | ml/kg/day | 24-41 | 22-30 |
| Osmolality | | 500-1200: | 50 min: |
| | | 50 min: | 3000 max. |
| | | 2400 max. | |
| Sediment: erythrocytes | | 0-5 | 0-5 |
| (per HPF) | | | |
| Sediment: leukocytes | | 0-5 | 0-5 |
| (per HPF) | | | |
| Sediment: casts (per HPF) | | 0 | 0 |
| Glucose/Ketones | | 0 | 0 |
| Bilirubin | | 0-trace | 0 |
| Calcium | mEq/L | 2-10 | |
| Creatinine | mg/dL | 100-300 | 110-280 |
| Chloride | mEq/L | 0-400 | |
| Magnesium | mg/kg/24 h | 1.7-3.0 | 3 |
| Phosphorus | mEq/L | 50-180 | |
| Potassium | mEq/L | 20-120 | |
| Sodium | mEq/L | 20-165 | |
| Urea Nitrogen | mg/kg/ 24 h | 140-2302 | 374-1872 |

*no normal range established in this laboratory [symbol]
indicates data missing or illegible when filed

It is noted here that while in some embodiments, a companion animal is started on the compositions when one or more variables are outside the normal range, in other embodiments, a companion animal with variables that fall within normal ranges may also be started on the compositions as described herein. In other words, compositions according to aspects of the present invention may be used to promote healthy hip, joint and kidney function in an animal having normal ranges for any of a variety of specific plasma, urinary, endocrine, and/or other variables. No limitation in this regard is therefore intended.

In certain embodiments, the supplement formulation of the compositions includes one or more active agents that promote healthy hip and joints, i.e., one or more healthy hip and joint active agents. In certain aspects, a given agent is considered to promote healthy hip and joints, and therefore be a healthy hip and joint active agent, if it results in some measurable improvement in hip and joint activity as compared to a control. Agents that promote healthy hip and joints include, but are not limited to, anti-inflammatory agents, including anti-inflammatory/pain-relief agents. In some instances, the one or more healthy hip and joint actives include an effective amount of Willow bark. In certain embodiments, only one part of the herb is used for the active component, e.g., the bark. In certain embodiments, the effective amount of Willow bark in the formulation is in an amount such that the companion animal ingests the equivalent of from 50 mg of dry Willow bark herb/kg body weight/day to 500 mg of dry Willow bark herb/kg body weight/day, including any subset of ranges or specific amount there-between. For example, in certain embodiments, the companion animal ingests the equivalent of from 100 mg dry Willow bark herb/kg body weight/day to 1000 mg Willow bark herb/kg body weight/day, the equivalent of from 250 mg dry Willow bark herb/kg body weight/day to 2500 mg Willow bark herb/kg body weight/day. For example, Willow bark may be present in the composition such that, when administered to the companion animal (e.g., according to a dosing schedule), the companion animal ingests the equivalent of 50 mg, 100 mg, 250 mg, 500 mg, 1 g, up to 2.5 g or more of dry Willow bark herb/kg body weight/day.

It is noted that a specific composition can contain any of a variety of different forms of Willow bark, not only the dry herb form. Therefore, an “equivalent” or “equivalent amount” of Willow bark in the dry herb form means that the total amount of Willow bark-derived actives present in the supplement formulation is equivalent to the actives present in the specified dry herb amount of Willow bark.

In certain embodiments, the one or more healthy hip and joint actives include an effective amount of Cat's claw. In certain embodiments, only one part of the herb is used for the active component, e.g., the root. In certain embodiments, the effective amount of Cat's claw in the formulation is in an amount such that the companion animal ingests the equivalent of from 50 mg of dry Cat's claw herb/kg body weight/day to 500 mg of dry Cat's claw herb/kg body weight/day, including any subset of ranges or specific amount there-between. For example, in certain embodiments, the companion animal ingests the equivalent of from 100 mg dry Cat's claw herb/kg body weight/day to 1000 mg Cat's claw herb/kg body weight/day, the equivalent of from 250 mg dry Cat's claw herb/kg body weight/day to 2500 mg Cat's claw herb/kg body weight/day. For example, Cat's claw may be present in the formulation such that, when administered to the companion animal (e.g., according to a dosing schedule), the companion animal ingests the equivalent of 50 mg, 100 mg, 250 mg, 500 mg, 1 g, up to 2.5 g or more of dry Cat's claw herb/kg body weight/day.

It is noted that a specific composition can contain any of a variety of different forms of Cat's claw, not only the dry herb form. Therefore, an “equivalent” or “equivalent amount” of Cat's claw in the dry herb form means that the total amount of Cat's claw-derived actives present in the composition is equivalent to the actives present in the specified dry herb amount of Cat's claw.

In certain embodiments, the compositions include a combination of Willow bark and Cat's claw such that the companion animal ingests the equivalent of the amounts of each dray herb as detailed above. Any combination of the equivalent ingested amounts of the dry herbs may be employed (i.e., the equivalent amounts of the dry herbs to be ingested may be selected independently from the amounts detailed above). No limitation in this regard is intended.

In certain embodiments, the supplements of the compositions include one or more additional actives effective in promoting healthy hip and joint function. Exemplary additional actives include, but are not limited to, Devil's Claw, Ashwagandha, Corydalis, Fish Oil, DHA, EPA, Glucosamine, Chondroitin Sulphate and any combination thereof.

In certain embodiments, the compositions are administered to the companion animal over time to treat, ameliorate, modify or prevent a hip and joint disease or dysfunction. The duration of administering the supplement formulations may last from a week, to a month, to months, to a year, to multiple years. During this time, the companion animal may be assessed for symptoms or risk factors to determine the efficacy of the composition in treating, preventing, ameliorating or modifying hip and joint disease or dysfunction. In certain embodiments, the results obtained from the assessment may be used to revise the supplement formulation dosage or composition. For example, if a hip and joint disease symptom of a companion animal is not reduced sufficiently in a first dosage timeframe, the supplement formulation may be adjusted, e.g., by increasing the frequency of administering the compositions containing supplement formulation to the companion animal or by providing a different compositions that differ with respect to supplement formulation having an increased actives profile (e.g., increased amounts of Willow bark and/or Cat's claw). In certain embodiments, e.g., where an animal is refusing to ingest the composition (e.g., due to color, texture, taste, odor, etc.), a different highly palatable backbone can be provided and administered to the companion animal (i.e., a composition in a form that the companion animal will be more likely to ingest). This is particularly true when the animal is sick because its sense of taste and smell is different from when it is healthy.

It is noted here that a composition can be administered to the companion animal in multiple different forms so that the most palatable backbone for the companion animal can be determined. In addition, multiple different backbones may be administered to the companion animal so that the animal does not tire of receiving the same composition over time. Such multiple different formulations may be provided at independent administration times or mixed and ingested at a single administration time. No limitation in this regard is intended.

In certain embodiments, the compositions are designed to be administered to the companion animal at intervals during the day to achieve the desired ingested dose, e.g., twice daily, three times daily, four times daily, five times daily, etc. In such embodiments, administering the composition to the companion animal is conducted multiple times/day.

In certain embodiments, the companion animal has a hip and joint disease or a risk factor for a hip and joint disease including, advanced age, large size and other risk factors including but not limited to inflammatory conditions. In certain embodiments, the companion animal is a dog or a cat or a horse.

In addition to the actives, the highly palatable compositions may include one or more additional ingredients, as desired. In certain embodiments, the additional ingredients are those that contribute to the highly palatable backbone. Such additional ingredients can serve to generate a backbone that is desirable to the companion animal for which it is intended, e.g., to have a desirable color, taste, texture, smell, hardness, softness, viscosity, mouthfeel, etc. In addition to additional ingredients that contribute to the highly palatable backbone, certain additional ingredients can provide other active components. Additional ingredients include an one or any combination of the following non-limiting examples (exemplary, non-limiting ranges for amounts of each additional ingredient are % by weight): Poultry Liver digest Spray dried (e.g., 30% by weight up to whatever amount is needed to balance the formula), Whey (e.g., 12% to 180% by weight), Glycerin (e.g., 10% to 14% by weight; humectant), malted barley flour (e.g., 10% to 14% by weight), fish oil mixed tocopherols (e.g., 2% to 5% by weight; source of omega-3 fatty acids), salts (e.g., 0.1% to 5% by weight), flavoring herbs and spices (e.g., 0.1% to 10% by weight), black malt extract (e.g., 1% to 10% by weight), pea protein (e.g., 1% to 4% by weight), brewers dried yeast (e.g., 1% to 4% by weight), lecithin (e.g., 0.5% to 3% by weight), Fish concentrate flavor (e.g., 1% to 2% by weight), glucono-delta lactone (e.g., 0.2% to 0.4% by weight), citric acid (e.g., 0.1% to 0.3% by weight), mixed tocopherols (e.g., 0.05% to 2% by weight), vitamins (e.g., 0.1% to 5% by weight), minerals (e.g., 0.1% to 5% by weight), Pro-bond 1580 (e.g., 1% to 8% by weight), natural flavor (1% to 8% by weight), fish oil (e.g., 1% to 8% by weight), C'Sens 9P 6446 DP (Internal Application; e.g., 1% to 5% by weight), C'Sens 9P 6446 DP (External application/coating; e.g., 0.5% to 3% by weight), caramel color (e.g., 0.3% to 1% by weight), Naturox (e.g., 0.05% to 0.3% by weight), etc. It is noted that the compositions can include other ingredients not listed above.

In certain embodiments, the composition lacks essential nutrient components, and thus is not meant to serve as a replacement for the companion animal's normal diet or to provide complete and balanced nutrition.

As described above, aspects of the subject invention include methods of supporting healthy hip and joint function (or treating, preventing, ameliorating or modifying a hip and joint disease or dysfunction) in a companion animal by administering the companion animal composition of the invention that includes a supplement formulation in combination with a highly palatable backbone, where the supplement formulation includes an effective amount of a healthy hip and joint active, e.g., Willow bark and/or Cat's claw. As such, healthy hip and joint compositions that find use in such methods are manufactured to provide such effective amounts of one or more active agents that supports healthy hip and joint function, e.g., Willow bark and/or Cat's claw. Because the compositions may be provided in multiples, e.g., multiple soft chews or tablets, the amount of an active agent(s) that supports healthy hip and joint function in each unit of the supplement formulation can vary widely.

For example, to provide the equivalent of 300 mg dry Willow bark herb/kg body weight/day to a companion animal, 5 soft chews each containing the equivalent of 20 mg dry herb can be administered to the animal 3 times a day (5 soft chews/dose×20 mg dry herb/soft chew×3 doses/day=300 mg dry herb/day).

Thus, in certain embodiments, compositions contain the equivalent of anywhere from 5 mg to 500 mg of Willow bark dry herb and/or the equivalent of anywhere from 5 mg to 500 mg Cat's claw dry herb, including any amount between these intervals. Therefore, compositions according to aspects of the invention include the equivalent of 5 mg, 10 mg, 20 mg, 50 mg, 100 mg, 200 mg, 400 mg, 500 mg, 600 mg, 700 mg, 800 mg, 900 mg, 1 g, 2.5 g, 5 g, 10 g, 20 g, 50 g, 100 g, 200 g, 400 g, etc., of Willow bark dry herb and/or Cat's claw dry herb (and any combination thereof, e.g., 500 mg Willow bark and 50 mg Cat's claw). In additional embodiments, the composition is provided in a single bulk amount that is to be administered to the companion animal in doses over time. For example, a composition can be provided as a liquid that is administered to the companion animal in doses over multiple days/weeks/months. In such embodiments, the compositions can include higher equivalent amounts of the Willow bark and/or Cat's claw dry herb than would be present in non-bulk forms.

The compositions may be produced in any convenient manner. In certain embodiments, a manufacturing process is employed in which extract powders or liquid extracts, e.g., of Willow bark and/or Cat's claw, are used. Such powders or extracts can be prepared in different ways which result in a concentrate that can result in a reduction in the inclusion amount of the active in question in the formulation (i.e., a powder or extract with a higher concentration of active than the dry herb can be used at a lower amount than the dry herb itself). For example, an extract powder made at a ratio of 10:1 would indicate that the extract would need to be included at one-tenth ( 1/10) the amount of the crude non-extracted powder of the dried herb. In this particular case, if the Willow Bark extract powder was used instead of the dried herb, then the dose of this ingredient would be 5 mg/kg/day to 50 g/kg/day instead of 50 mg/kg/day to 500 g/kg/day as described above. However, it is noted that, as described above, the 5 mg/kg/day to 50 g/kg/day of the extract powder in the composition is the equivalent of 50 mg/kg/day to 500 g/kg/day of the dry herb. Similarly, a decoction of 5-30 g per cup of water administered three times a day at a rate of ¼ to ½ cup per 10 kg can be equivalent to approximately 375 mg/kg/day to 4.5 g/kg/day. A tincture (e.g., in 25-35% ethanol) of 1:2-1:3 given three times a day at 1-2 ml per 10 kg can be equivalent to approximately 100 mg/kg/day to 300 mg/kg/day. The approximate amounts provided to the companion animal in decoctions and tinctures (as well as other formulations) may vary depending on the efficiency of the extraction process employed in making the supplement formulation.

In certain embodiments, the composition is a tablet that can be dissolved in a liquid (e.g., water) prior to ingestion. Such tablets may be “fizzy tablets”, or effervescent tablets, that carbonate the liquid upon dissolving.

The healthy hip and joint compositions, e.g., as described herein, may be provided to the individual administering to the companion animal, and/or to a third person providing the composition to the individual, in any of a variety of convenient form of packaging, including a bag, box, container, cup, bowl, bottle, and the like, where in some embodiments the package is reusable, re-sealable, or refillable. The packaging may have multiple, separate sub-compartments that are configured to hold a specific amount of the supplement formulations (e.g., be packaged in unit dosage form). The amount in each sub-compartment may include any number of individual formulation units (e.g., any number of soft chews or tablets, e.g., 1, 2, 3, 4, 5, 6, 7, 8, 9, 10, 20, 30, 40, etc., and any number in between) to achieve a desired dosage of actives in the supplement formulation (e.g., a specific dosage or amount of Willow bark and/or Cat's claw actives). Each sub-compartment may be configured for consumption within a specified time period, e.g., five times a day, four times a day, three times a day, two times a day, every day, every two days, every three days, every four days, every five days, every six days, weekly, bi-weekly, monthly, etc. The packaging may have printed thereon an administration schedule that specifies when or how much of the supplement formulation to feed the companion animal, where the administration schedule may be split into amounts to feed the companion animal based on weight of the companion animal. In addition, when the packaging has multiple sub-compartments, each sub-compartment may have printed thereon the day and/or time of day to feed the enclosed supplement formulation to the companion animal (e.g., similar to a multi-compartment pill box which lists the days of the week that the pills inside should be taken by a human individual).

In certain embodiments, the composition is provided with instructions for use according to the methods of the present invention, aspects of which are described above.

As mentioned above, another progressive degenerative condition of interest is renal disease or risk of developing renal disease. As such, in certain embodiments, the companion animal has a renal disease or a risk factor for a renal disease including, but not limited to, chronic renal disease, glomerular disease, infectious disease, neoplasia, inflammatory disease, tubulointerstitial disease, urinary tract infection, urinary stones, obstructions of lower urinary tract, exposure to toxic drugs, sequelae of acute renal failure, diabetes, hypercalcemia, heart failure, treatment with aminoglycosides, sulfonamides, polymyxin B, amphotericin and chemotherapeutic drugs, high blood pressure, high level of proteinuria, and poor glycemic control in diabetes mellitus. In certain embodiments, the companion animal is a dog or a cat or a horse.

In certain embodiments, the supplement formulation of the compositions includes one or more active agents that effective in treating a renal condition, i.e., one or more renal condition agents. In certain aspects, a given agent is considered to be effective in treating a renal condition, and therefore be a renal condition active agent, if it results in some measurable improvement in renal activity as compared to a control. Agents that are effective in treating renal conditions include, but are not limited to, anti-inflammatory agents, including anti-inflammatory/hypotensive agents.

In certain of these embodiments, the supplement formulation includes an effective amount of Rehmannia. In certain embodiments, only one part of the herb is used for the active component, e.g., the root. In certain embodiments, the effective amount of Rehmannia in the formulation is in an amount such that the companion animal ingests the equivalent of from 50 mg of dry Rehmannia herb/kg body weight/day to 500 g of dry Rehmannia herb/kg body weight/day, including any subset of ranges or specific amount there-between. For example, in certain embodiments, the companion animal ingests the equivalent of from 100 mg dry Rehmannia herb/kg body weight/day to 100 g Rehmannia herb/kg body weight/day, the equivalent of from 500 mg dry Rehmannia herb/kg body weight/day to 50 g Rehmannia herb/kg body weight/day, or the equivalent of from 10 g dry Rehmannia herb/kg body weight/day to 25 g Rehmannia herb/kg body weight/day. For example, Rehmannia may be present in the formulation such that, when administered to the companion animal (e.g., according to a dosing schedule), the companion animal ingests the equivalent of 50 mg, 100 mg, 250 mg, 500 mg, 1 g, 2.5 g, 5 g, 10 g, 25 g, 50 g, 100 g, 250 g, up to 500 g or more of dry Rehmannia herb/kg body weight/day.

It is noted that a specific supplement formulation can contain any of a variety of different forms of Rehmannia, not only the dry herb form. Therefore, an “equivalent” or “equivalent amount” of Rehmannia in the dry herb form means that the total amount of Rehmannia-derived actives present in the supplement formulation is equivalent to the actives present in the specified dry herb amount of Rehmannia.

In certain embodiments, the supplement formulation includes an effective amount of Astragalus. In certain embodiments, only one part of the herb is used for the active component, e.g., the root. In certain embodiments, the effective amount of Astragalus in the formulation is in an amount such that the companion animal ingests the equivalent of from 50 mg of dry Astragalus herb/kg body weight/day to 500 g of dry Astragalus membranaceus herb/kg body weight/day, including any subset of ranges or specific amount there-between. For example, in certain embodiments, the companion animal ingests the equivalent of from 100 mg dry Astragalus herb/kg body weight/day to 100 g Astragalus herb/kg body weight/day, the equivalent of from 500 mg dry Astragalus herb/kg body weight/day to 50 g Astragalus herb/kg body weight/day, or the equivalent of from 10 g dry Astragalus herb/kg body weight/day to 25 g Astragalus herb/kg body weight/day. For example, Astragalus may be present in the formulation such that, when administered to the companion animal (e.g., according to a dosing schedule), the companion animal ingests the equivalent of 50 mg, 100 mg, 250 mg, 500 mg, 1 g, 2.5 g, 5 g, 10 g, 25 g, 50 g, 100 g, 250 g, up to 500 g or more of dry Astragalus herb/kg body weight/day.

It is noted that a specific supplement formulation can contain any of a variety of different forms of Astragalus, not only the dry herb form. Therefore, an “equivalent” or “equivalent amount” of Astragalus in the dry herb form means that the total amount of Astragalus-derived actives present in the supplement formulation is equivalent to the actives present in the specified dry herb amount of Astragalus.

In certain embodiments, the supplement formulations include a combination of Rehmannia and Astragalus such that the companion animal ingests the equivalent of the amounts of each dray herb as detailed above. Any combination of the equivalent ingested amounts of the dry herbs may be employed (i.e., the equivalent amounts of the dry herbs to be ingested may be selected independently from the amounts detailed above). No limitation in this regard is intended.

In certain embodiments, the supplement formulations include one or more additional actives effective in promoting healthy kidney function. Exemplary additional actives include, but are not limited to, nettle seed, cordyceps, marshmallow, Siberian ginseng, angelica, ligusticum, Tripterygium wilfordii (and its active triptolide), rhubarb and any combination thereof.

In certain embodiments, the formulations are administered to the companion animal over time to treat, ameliorate, modify or prevent a renal disease or dysfunction. The duration of administering the supplement formulations may last from a week, to a month, to months, to a year, to multiple years. During this time, the companion animal may be assessed for symptoms or risk factors to determine the efficacy of the supplement formulation in treating, preventing, ameliorating or modifying renal disease or dysfunction. In certain embodiments, the results obtained from the assessment may be used to revise the supplement formulation dosage or composition. For example, if a renal disease symptom of a companion animal is not reduced sufficiently in a first dosage timeframe, the supplement formulation may be adjusted, e.g., by increasing the frequency of administering the supplement formulation to the companion animal or by providing a different supplement formulation having an increased actives profile (e.g., increased amounts of Rehmannia and/or Astragalus). In certain embodiments, if an animal is refusing to ingest the supplement formulation (e.g., due to color, texture, taste, odor, etc.), a different highly palatable backbone can be provided and administered to the companion animal (i.e., a supplement formulation in a form that the companion animal will be more likely to ingest). This is particularly true when the animal is sick because its sense of taste and smell is different from when it is healthy.

It is noted here that a supplement formulations can be administered to the companion animal in multiple different forms so that the most palatable backbone for the companion animal can be determined. In addition, multiple different backbones may be administered to the companion animal so that the animal does not tire of receiving the same supplement formulation over time. Such multiple different formulations may be provided at independent administration times or mixed and ingested at a single administration time. No limitation in this regard is intended.

In certain embodiments, the supplement formulations are designed to be administered to the companion animal at intervals during the day to achieve the desired ingested dose, e.g., twice daily, three times daily, four times daily, five times daily, etc. In such embodiments, administering the supplement formulation to the companion animal is conducted multiple times/day.

In addition to the actives, the highly palatable supplement formulations include additional ingredients. In certain embodiments, the additional ingredients are those that contribute to the highly palatable backbone. Such additional ingredients can serve to generate a backbone that is desirable to the companion animal for which it is intended, e.g., to have a desirable color, taste, texture, smell, hardness, softness, viscosity, mouth feel, etc. In addition to additional ingredients that contribute to the highly palatable backbone, certain additional ingredients can provide other active components. Additional ingredients include one or any combination of the following non-limiting examples (exemplary, non-limiting ranges for amounts of each additional ingredient are % by weight): Poultry Liver digest Spray dried (e.g., 30% by weight up to whatever amount is needed to balance the formula), Whey (e.g., 12% to 180% by weight), Glycerin (e.g., 10% to 14% by weight; humectant), malted barley flour (e.g., 10% to 14% by weight), fish oil mixed tocopherols (e.g., 2% to 5% by weight; source of omega-3 fatty acids), salts (e.g., 0.1% to 5% by weight), flavoring herbs and spices (e.g., 0.1% to 10% by weight), black malt extract (e.g., 1% to 10% by weight), pea protein (e.g., 1% to 4% by weight), brewers dried yeast (e.g., 1% to 4% by weight), lecithin (e.g., 0.5% to 3% by weight), Fish concentrate flavor (e.g., 1% to 2% by weight), glucono-delta lactone (e.g., 0.2% to 0.4% by weight), citric acid (e.g., 0.1% to 0.3% by weight), mixed tocopherols (e.g., 0.05% to 2% by weight), vitamins (e.g., 0.1% to 5% by weight), minerals (e.g., 0.1% to 5% by weight), Pro-bond 1580 (e.g., 1% to 8% by weight), natural flavor (1% to 8% by weight), fish oil (e.g., 1% to 8% by weight), C'Sens 9P 6446 DP (Internal Application; e.g., 1% to 5% by weight), C'Sens 9P 6446 DP (External application/coating; e.g., 0.5% to 3% by weight), caramel color (e.g., 0.3% to 1% by weight), Naturox (e.g., 0.05% to 0.3% by weight), etc. It is noted that the supplement formulations can include other ingredients not listed above.

In certain embodiments, the supplement formulation lacks essential nutrient components, and thus is not meant to serve as a replacement for the companion animal's normal diet.

As described above, aspects of the subject invention include methods of supporting healthy kidney function (or treating, preventing, ameliorating or modifying a renal disease or dysfunction) in a companion animal by administering the companion animal a supplement formulation on a highly palatable backbone that includes an effective amount of an active that supports healthy renal function, e.g., Rehmannia and/or Astragalus. As such, highly palatable supplement formulations that find use in such methods are manufactured to provide such effective amounts of an active that supports healthy renal function, e.g., Rehmannia and/or Astragalus. Because the supplement formulations may be provided in multiples, e.g., multiple kibbles or treats, the amount of an active that supports healthy renal function in each unit of the supplement formulation can vary widely.

For example, to provide the equivalent of 300 mg dry Rehmannia herb/kg body weight/day to a companion animal, 5 kibbles each containing the equivalent of 20 mg dry herb can be administered to the animal 3 times a day (5 kibbles/dose×20 mg dry herb/kibble×3 doses/day=300 mg dry herb/day).

Thus, in certain embodiments, supplement formulations contain the equivalent of anywhere from 5 mg to 500 g of Rehmannia dry herb and/or the equivalent of anywhere from 5 mg to 500 g Astragalus dry herb, including any amount between these intervals. Therefore, supplement formulations according to aspects of the invention include the equivalent of 5 mg, 10 mg, 20 mg, 50 mg, 100 mg, 200 mg, 400 mg, 500 mg, 600 mg, 700 mg, 800 mg, 900 mg, 1 g, 5 g, 10 g, 20 g, 50 g, 100 g, 200 g, 400 g, 500 g, etc., of Rehmannia dry herb and/or Astragalus dry herb (and any combination thereof, e.g., 500 mg Rehmannia and 50 mg Astragalus). In additional embodiments, the supplement formulation is provided in a single bulk amount that is to be administered to the companion animal in doses over time. For example, a supplement formulation can be provided as a liquid that is administered to the companion animal in doses over multiple days/weeks/months. In such embodiments, the supplement formulation can include higher equivalent amounts of the Rehmannia and/or Astragalus dry herb than would be present in non-bulk forms.

The supplement formulations may be generated in any convenient manner. In certain embodiments, the supplement formulation employs a manufacturing processes in which extract powders or liquid extracts, e.g., of Rehmannia and/or Astragalus, are employed. Such powders or extracts can be prepared in different ways which result in a concentrate that can result in a reduction in the inclusion amount of the active in question in the formulation (i.e., a powder or extract with a higher concentration of active than the dry herb can be used at a lower amount than the dry herb itself). For example, an extract powder made at a ratio of 10:1 would indicate that the extract would need to be included at one-tenth ( 1/10) the amount of the crude non-extracted powder of the dried herb. In this particular case, if the Rhemannia extract powder was used instead of the dried herb, then the dose of this ingredient would be 5 mg/kg/day to 50 g/kg/day instead of 50 mg/kg/day to 500 g/kg/day as described above. However, it is noted that, as described above, the 5 mg/kg/day to 50 g/kg/day of the extract powder in the formulation is the equivalent of 50 mg/kg/day to 500 g/kg/day of the dry herb. Similarly, a decoction of 5-30 g per cup of water administered three times a day at a rate of ¼ to ½ cup per 10 kg can be equivalent to approximately 375 mg/kg/day to 4.5 g/kg/day. A tincture (e.g., in 25-35% ethanol) of 1:2-1:3 given three times a day at 1-2 ml per 10 kg can be equivalent to approximately 100 mg/kg/day to 300 mg/kg/day. The approximate amounts provided to the companion animal in decoctions and tinctures (as well as other formulations) may vary depending on the efficiency of the extraction process employed in making the supplement formulation.

In certain embodiments, the highly palatable supplement formulation is a tablet that can be dissolved in a liquid (e.g., water) prior to ingestion. Such tablets may be “fizzy tablets”, or effervescent tablets, that carbonate the liquid upon dissolving.

The highly palatable supplement formulation may be provided to the individual administering to the companion animal, and/or to a third person providing the supplement formulation to the individual, in any of a variety of convenient form of packaging, including a bag, box, container, cup, bowl, bottle, and the like, where in some embodiments the package is reusable, re-sealable, or refillable. The packaging may have multiple, separate sub-compartments that are configured to hold a specific amount of the supplement formulations (e.g., be packaged in unit dosage form). The amount in each sub-compartment may include any number of individual formulation units (e.g., any number of kibbles or treats, e.g., 1, 2, 3, 4, 5, 6, 7, 8, 9, 10, 20, 30, 40, etc., and any number in between) to achieve a desired dosage of actives in the supplement formulation (e.g., a specific dosage or amount of Rehmannia glutinosa and/or Astragalus membranaceus actives). Each sub-compartment may be configured for consumption within a specified time period, e.g., five times a day, four times a day, three times a day, two times a day, every day, every two days, every three days, every four days, every five days, every six days, weekly, bi-weekly, monthly, etc. The packaging may have printed thereon an administration schedule that specifies when or how much of the supplement formulation to feed the companion animal, where the administration schedule may be split into amounts to feed the companion animal based on weight of the companion animal. In addition, when the packaging has multiple sub-compartments, each sub-compartment may have printed thereon the day and/or time of day to feed the enclosed supplement formulation to the companion animal (e.g., similar to a multi-compartment pill box which lists the days of the week that the pills inside should be taken by a human individual).

In certain embodiments, the supplement formulation is provided with instructions for use according to the methods of the present invention, aspects of which are described above.

There are many different highly palatable backbones that may be employed in practicing aspects of the present invention, and as such, no limitation in this regard is intended. In general, the highly palatable backbone will be one known or predicted to be desired by the companion animal for ingestion. Desirable characteristics include smell, taste, texture, color, odor, shape, hardness/softness, size, viscosity, mouthfeel, etc. In a particular embodiment the shape of the edible backbone can be designed to provide the animal with “play” potential while it eats the supplement formulation and more closely satisfy the natural “kill” instinct of the animal, adding to the overall palatability of the supplement formulation (e.g., the formulation can be in the form of a ball, an oval or a cylinder). Other shapes that induce the natural kill and/or play instinct in the animal may be employed.

The backbone can be a solid, liquid or gel composition, including a soft chew, tablet, drink, etc. In certain embodiments, the composition is provided in a form that is mixed with a carrier prior to ingestion. For example, the composition may be provided in tablet or liquid form that is to be dissolved in or mixed with water prior to ingestion by the companion animal. In another example, the composition may be in a powder, liquid or gel formulation that can be sprinkled on or applied to a companion animal's food. For example, the composition may be a flavored gravy composition that is applied over the top of the companion animal's dry food. In a certain embodiment the actives combined with the edible backbone are encapsulated for the purpose of stabilization during manufacture, storage and/or for the purpose of reducing bad odors or smells. In addition, certain embodiments of the composition have the active(s) encapsulated or are otherwise manufactured to have controlled release of the active(s) over time. In other words, certain of the supplement formulations found in the compositions are time-released supplement formulations (also called sustained-release (SR), sustained-action (SA), extended-release (ER, XR, or XL), time-release or timed-release, controlled-release (CR), modified release (MR), or continuous-release (CR or Contin)). Time-released supplement formulations can often be taken less frequently than instant-release supplement formulations, often providing for steadier levels of the active(s) in the bloodstream of the companion animal. Any convenient time-release technology may be employed in the supplement formulations described herein.

### Combinations

It is appreciated that certain aspects of the invention, which are, for clarity, described in the context of separate embodiments, may also be provided in combination in a single embodiment. Conversely, various aspects of the invention, which are, for brevity, described in the context of a single embodiment, may also be provided separately or in any suitable sub-combination. All combinations of the embodiments pertaining to the ingredients in the subject companion animal formulations are specifically embraced by the present invention and are disclosed herein just as if each and every combination was individually and explicitly disclosed. In addition, all sub-combinations of the ingredients in the subject companion animal formulations listed in the embodiments describing such variables are also specifically embraced by the present invention and are disclosed herein just as if each and every such sub-combination of ingredients was individually and explicitly disclosed herein.

In order that the invention may be more readily understood, reference is made to the following examples, which are intended to be illustrative of the invention, but are not intended to be limiting in scope.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Pet Hip and Joint Active Formula: Willow Bark 1×; Cat's Claw: 1×

TABLE 2
Dog Hip and joint Formulation
(numbers are % by weight)

| Ingredient | % |
|---|---|
| potato flour | 25.36% |
| spray dried chicken and chicken broth | 16.00% |
| glycerin | 15.00% |
| fish sauce natural flavor | 8.00% |
| whey | 5.00% |
| molasses | 5.00% |
| D’tech 8p6998 | 5.00% |
| Pro Bon 1580d | 4.00% |
| Fish Oil | 4.00% |
| Pork Broth Spray dried | 3.00% |
| Willow Bark extract 25% salicin | 2.09% |
| Cats Claw root extract 4:1 | 2.09% |
| Brewer's Dried Yeast | 2.00% |
| Soy Lecithin | 1.00% |
| Caramel Color | 1.00% |
| Phosphoric Acid | 0.75% |
| Sorbic Acid | 0.30% |
| Citric Acid | 0.30% |
| Naturox | 0.10% |
| Talin | 0.01% |

Formula assumptions for Table 1:

Each formulation is based on 4 g soft chew.

Administered on the basis of 1 chew/day/30 lb body weight:

Each soft chew has 84 mg of each of Cat's claw and Willow Bark: 84 mg in 4000 mg (each piece being 4 g=4000 mg) is 2.1% each of Cat's claw and Willow Bark in the formulation.

### Pet Renal Active Formula: Rhemannia 1×; Astragalus: 1-2×

TABLE 2
Exemplary Cat Renal Formulation (numbers are % by weight)

| | Based on 3 | Based on 8 | |
|---|---|---|---|
| | OZ bag and if | OZ bag and if | Additional |
| Ingredient | 3 pieces/day | 9 pieces/day | Formulation |
| Poultry Liver digest | 39.7 | 44.2 | 39.65 |
| Spray dried | | | |
| Whey | 15 | 15 | 15 |
| Glycerin | 12 | 12 | 15 |
| malted barley flour | 12 | 12 | — |
| fish oil mixed | 4 | 4 | — |
| tocopherols | | | |
| black malt extract | 3 | 3 | — |
| pea protein | 2 | 2 | — |
| brewers dried yeast | 2 | 2 | — |
| lecithin | 1.5 | 1.5 | 1.75 |
| Fish concentrate flavor | 1.5 | 1.5 | — |
| glucono-delta lactone | 0.3 | 0.3 | 0.5 |
| citric acid | 0.2 | 0.2 | 0.3 |
| mixed tocopherols | 0.1 | 0.1 | — |
| Pro-bond 1580 | — | — | 5 |
| Natural flavor | — | — | 4.5 |
| Fish oil | — | — | 4 |
| C’Sens 9P 6446 DP | — | — | 3 |
| (Internal Application) | | | |
| C’Sens 9P 6446 DP | — | — | 1.5 |
| (External application- | | | |
| coating) | | | |
| Caramel Color | — | — | 0.7 |
| Naturox | — | — | 0.1 |
| | Active % | Active % | Active % |
| Astragalus 1:10 extract | 4.5 | 1.5 | 6 |
| powder | | | |
| Rhemannia 1:10 extract | 2.25 | 0.75 | 3 |
| powder | | | |

Formula assumptions for Table 2:

Each formulation is based on 1 g/piece.

3 oz. bag administered on the basis of 3 pieces/day:

Each piece has 45 mg Astragalus: 45 mg in 1000 mg (each piece being 1 g=1000 mg) is 4.5% Astragalus in the formulation.

Each piece has 22.5 mg Rhemannia; 22.5 in 1000 mg (each piece being 1 g=1000 mg) is 2.25% Rhemannia in the formulation.

8 oz. bag administered on the basis of 9 pieces/day:

Each piece has 15 mg Astragalus: 15 mg in 1000 mg (each piece being 1 g=1000 mg) is 1.5% Astragalus in the formulation.

Each piece has 7.5 mg Rhemannia; 7.5 mg in 1000 mg (each piece being 1 g=1000 mg) is 0.75% Rhemannia in the formulation.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of maintaining health and/or treating a progressive degenerative condition in a companion animal, the method comprising:
administering to a companion animal a composition comprising:
(a) a supplement formulation comprising a health maintenance and/or progressive degenerative condition active agent in an amount effective to maintain health and/or treat the progressive degenerative condition; and
(b) a highly palatable backbone;
in a manner sufficient to maintain health and/or treat the companion animal for the progressive degenerative condition.

2. The method according to claim 1, wherein the active agent comprises a naturally occurring active agent.

3. The method according to claim 2, wherein the naturally occurring active agent is a plant or derivative thereof.

4. The method according to claim 3, wherein the plant is an herb.

5. The method according to claim 1, wherein the active agent exhibits anti-inflammatory activity.

6. The method according to claim 5, wherein the active agent exhibits anti-inflammatory activity and pain-relief activity.

7. The method according to claim 5, wherein the active agent exhibits anti-inflammatory activity and hypotensive activity.

8. The method according to claim 1, wherein the supplement formulation comprises one or more active agents selected from the group consisting of: Willow bark, Cat's claw, Rehmannia, Astragalus, nettle seed, marshmallow, Siberian ginseng, Angelica, ligusticum, triptolide, rhubarb, Devil's claw, Corydalis, Ashwagandha, Fish Oil, DHA, EPA, Gluconsamine, Condroitin Sulfat, Cordyceps and any combination thereof.

9. The method of claim 1, wherein the supplement formulation comprises Willow bark.

10-13. (canceled)

14. The method claim 1, wherein the supplement formulation comprises Cat's claw.

15-18. (canceled)

19. The method of claim 1, wherein the supplement formulation comprises Rehmannia.

20-23. (canceled)

24. The method of claim 1, wherein the supplement formulation comprises Astragalus.

25-29. (canceled)

30. The method of claim 1, wherein the companion animal is selected from the group consisting of: a dog and a cat or a horse.

31. The method of claim 1, wherein the highly palatable backbone is selected from the group consisting of: a soft chew, a tablet, and a drink.

32. The method of claim 1, wherein the composition lacks essential nutrient components.

33. The method of claim 1, wherein the companion animal has a DJD or a risk factor for a DJD selected from the group consisting of: chronic hip and joint disease, inflammatory disease, advanced age, large size, obesity or any combination thereof.

34. The method of claim 33, wherein the supplement formulation is in the form of a liquid extract, a decoction, an isolated or purified fraction, an extract powder, a tincture, or combinations thereof.

35. The method of claim 1, wherein the companion animal has a renal disease or a risk factors for a renal disease selected from the group consisting of: chronic renal disease, glomerular disease, infectious disease, neoplasia, inflammatory disease, tubulointerstitial disease, urinary tract infection, urinary stones, obstructions of lower urinary tract, exposure to toxic drugs, sequelae of acute renal failure, diabetes, hypercalcemia, heart failure, treatment with aminoglycosides, sulfonamides, polymyxin B, amphotericin and chemotherapeutic drugs, high blood pressure, high level of proteinuria, and poor glycemic control in diabetes mellitus.

36-40. (canceled)

41. A composition configured for administration to a companion animal, the composition comprising:
(a) a supplement formulation comprising a health maintenance and/orprogressive degenerative condition active agent in an amount effective to maintain health and/or treat the progressive degenerative condition; and
(b) a highly palatable backbone.

42. The composition according to claim 41, wherein the active agent comprises a naturally occurring active agent.

43-70. (canceled)
